(19) United States
(12) Patent Application Publication
(10) Pub. No.: US 2006/0269597 A1
(43) Pub. Date: Nov. 30, 2006

(54) Tannate Compositions and Methods of Use for the Treatment of Respiratory Tract Conditions

(76) Inventor: Jeffrey H. Ping, Braselton, GA (US)

Correspondence Address: SUTHERLAND ASBILL & BRENNAN LLP, 999 PEACHTREE STREET, N.E., ATLANTA, GA, 30309, US

(21) Appl. No.: 11/379,990

(22) Filed: Apr. 24, 2006

Related U.S. Application Data
(60) Provisional application No. 60/674,181, filed on Apr. 22, 2005.

(51) Int. Cl. A61K 31/205; A61K 9/64

(52) U.S. Cl. 424/456; 514/554

(57) ABSTRACT

Compositions comprising essentially of methscopolamine tannate alone or in combination with other tannate compounds which are effective when administered orally for the symptomatic relief of symptoms associated with upper respiratory tract conditions such as the common cold, sinusitis, allergic rhinitis, and other upper respiratory tract conditions.

### CROSS REFERENCES TO RELATED APPLICATIONS

This present application claims the priority benefit of U.S. Provisional Patent Application Ser. No. 60/674,181 filed on Apr. 22, 2005, the entire contents of which are hereby incorporated by reference.

### FIELD OF INVENTION

The invention relates generally to novel tannate compositions and their use in methods for the treatment of upper respiratory symptoms associated with respiratory tract infections or conditions. In particular, the invention provides novel compositions with antihistaminic, decongestant, and anticholinergic properties.

### BACKGROUND

Tannate compositions are widely used for the treatment of upper respiratory symptoms associated with respiratory tract conditions such as the common cold, allergic rhinitis, bronchial asthma, acute and chronic bronchitis, and sinusitis as well as allergic skin reactions including urticaria and angioedema. Such tannate compositions consist of various combinations of active ingredients in the tannate form from the antihistaminic, decongestant, anticholinergic, expectorant, and/or antitussive classes.

Chlorpheniramine competitively inhibits histamine at H₁ receptor sites, leading to vascular, respiratory, and gastrointestinal smooth muscle constriction preventing histamine mediated increase in vascular permeability, pruritis, and sneezing. Chemically, chlorpheniramine is know as 3-(p-chlorophenyl)-3-(2-pyridyl)-N,N-dimethylpropylamine a sythentic, optically active, racemic amine.

Phenylephrine is a decongestant that causes vasoconstriction via the activation of post-junctional α-adrenergic receptors located on the pre-capillary and post-capillary blood vessels of the nasal mucosa. Activation of these receptors occurs directly by binding of phenylephrine or indirectly by binding of norepinephrine released from sympathetic nerve endings in response to phenylephrine. Vasoconstriction in the nasal mucous membrane leads to improved drainage.

Chemically, phenylephrine is known as (−)-m-hydroxy-α-[(methylamino)-methyl]benzyl alcohol and is available in the levorotatory isomer.

Methscopolamine nitrate is a quaternary ammonium derivative of the anticholinergic scopolamine. Its anti-muscarinic effect inhibits salivary and bronchial secretions. Methscopolamine nitrate does not exhibit the central actions of other belladonna alkaloids due to its lack of ability to cross the blood-brain barrier. Chemically, methscopolamine nitrate is known as 3-Oxa-9-azoniatricyclo[3.3.1.0^{2,4}]nonane, 7-(3-hydroxy-1-oxo-2-phenylpropoxy)-9,9dimethyl-nitrate,[7(S)-(1α,2β,4β,5α,7β).

Decongestants and antihistaminics in the form of their tannate salts are very desirable because such salts are generally stable and may be combined in such form without any untoward side effects.

Tannate salts are typically prepared by reacting the drug free base with tannic acid in the presence of a volatile solvent, such as isopropanol or water, and then vacuum or freeze dried. Reaction variables such as mixing time and temperatures vary depending on the drug molecule and solvent used. Other methods of tannate preparation include the mixing of solid free base with solid tannic acid under heated conditions until completely converted to the tannate salt. Also, various in-situ methods are employed to convert compounds from their more commonly available salt forms such as citrate, hydrochloride, or maleate to the tannate salt as a part of the dosage form processing.

A considerable number of tannic acids occur in nature. Chemically, these acids are described as polymers of different hydroxybenzoic acids. Generally, when the term tannic acid is employed, as in the present case, the acid referred to is gallotannic acid. The internal ester of gallic acid also frequently referred to as tannin.

Tannic acid consists of an amorphous powder, glistening scales, or spongy masses varying in color from yellowish-white to light brown. Tannic acid is very soluble in water or alcohol. Commercially available, tannic acid, also known as tannin, has a complex non-uniform chemistry, usually contains from about 5% to about 10% water by weight, has a molecular weight of about 1700, and is typically produced from Turkish or Chinese nutgall.

### SUMMARY OF THE INVENTION

The present invention provides therapeutic compositions for the treatment of symptoms associated with the common cold, sinusitis, allergic rhinitis, respiratory congestion and other upper respiratory tract conditions as well as symptoms of allergic skin reactions such as urticaria and angioedema, in warm blooded animals in need of such treatment, said composition comprising pharmaceutically effective amounts of an anticholinergic compound, alone or in combination with an antihistamine, decongestant, or combinations thereof. In certain embodiments, the composition is a chewable tablet form or an orally disintegrating tablet form. In other embodiments, the composition is in suspension form. In yet other embodiments, the composition is in a liquid filled hard gelatin capsule form or a soft gelatin capsule form.

The present invention also provides that in certain preferred embodiments, the anticholinergic compound is methscopolamine tannate. The present invention also provides that the antihistamine may be selected from the group consisting of chlorpheniramine tannate, dexchlorpheniramine tannate, brompheniramine tannate, dexbrompheniramine tannate, carbinoxamine tannate, pyrilamine tannte, and diphenhydramine tannate. The invention further provides that in some embodiments, the decongestant is selected from the group consisting of phenylephrine tannate and pseudoephedrine tannate.

The present invention further provides that in certain embodiments the composition comprises pharmaceutically effective amounts of about 2 to 12 mg chlorpheniramine tannate, about 2 to 50 mg phenylephrine tannate, and about 0.5 to 10 mg methscopolamine tannate. In another embodiment, the composition comprises pharmaceutically effective amounts of about 3.5 mg chlorpheniramine tannate, about 25 mg phenylephrine tannate, and about 3 mg methscopolamine tannate.

The present invention provides methods for treating and relieving symptoms associated with a condition in a warm-blooded animal over an extended period of time, wherein the condition is selected from a group consisting of the common cold, sinusitis, allergic rhinitis, respiratory congestion, other upper respiratory tract conditions, allergic skin reactions, urticaria, and angioedema, and wherein the method comprises orally administering to a warm-blooded animal in need of such treatment a pharmaceutically effective amount of a composition comprising pharmaceutically effective amounts of an anticholinergic compound, alone or in combination with an antihistamine, decongestant, or combination thereof. In certain embodiments of these methods, the anticholinergic compound is methscopolamine tannate. In certain embodiments of these methods, the antihistamine is selected from the group consisting of chlorpheniramine tannate, dexchlorpheniramine tannate, brompheniramine tannate, dexbrompheniramine tannate, carbinoxamine tannate, pyrilamine tannte, and diphenhydramine tannate. In certain embodiments of these methods, the decongestant is selected from the group consisting of phenylephrine tannate and pseudoephedrine tannate.

The present invention also provides that in some embodiments of these methods, the composition comprises pharmaceutically effective amounts of about 2 to 12 mg chlorpheniramine tannate, about 2 to 50 mg phenylephrine tannate, and about 0.5 to 10 mg methscopolamine tannate. The present invention also includes methods wherein the composition comprises pharmaceutically effective amounts of about 3.5 mg chlorpheniramine tannate, about 25 mg phenylephrine tannate, and about 3 mg methscopolamine tannate.

The present invention further provides that in some embodiments of these methods, the composition is in a chewable tablet form or an orally disintegrating tablet form. In other embodiments, the composition is in a liquid filled hard gelatin capsule form or a soft gelatin capsule form. In yet other embodiments of these methods, the composition is in a suspension form.

### DETAILED DESCRIPTION

The present invention may be understood more readily by reference to the following detailed description and the Examples included herein. However, before the present compositions and methods are disclosed and described, it is to be understood that this invention is not limited to specific conditions or methods, etc., as such may, of course, vary, and the numerous modifications and variations therein will be apparent to those skilled in the art. It is also to be understood that as used in the specification and in the claims, “a” or “an” can mean one or more, depending upon the context in which it is used. Thus, for example, reference to “a compound” can mean that at least one compound can be utilized.

It has now been found that the novel combination of an antihistamine such as but not limited to chlorpheniramine tannate, a decongestant such as but not limited to phenylephrine tannate, and an anticholinergic such as but not limited to methscopolamine tannate produces a composition having antihistaminic, sympathomimetic, decongestant, and anticholinergic properties superior to the use of one of the tannate compounds alone.

It is believed that tannate salts of active agents provide therapeutic activity for longer time periods. In effect, the inclusion of an active agent in a tannate salt form extends the release profile of the active agent, and there is less spiking in the pharmacological effect of the active agent. This leads to better compliance by the patient in that the active agent in the tannate salt form does not need to be given as often and there are fewer side effects, particularly from over dosage effects.

The tannate compositions of the present invention can be made by methods know to those skilled in the art. Preparations of tannate compounds in a very pure form are taught in U.S. Pat. Nos. 5,599,846 and 5,663,415 to Chopdekar et al., which are herein incorporated in their entireties.

The tannate compositions described herein are designed to be taken twice a day in order to utilize the prolonged therapeutic action of the tannate compounds combined with the immediate action of methscopolamine tannate. The action of chlorpheniramine tannate, phenylephrine tannate, and/or methscopolamine tannate may be utilized collectively alone or in combination with the prolonged action of other compounds, either tannate or non-tannate in nature, or the immediate action of other compounds. The compositions of the present invention may be prepared for oral administration in the form of powders, capsules, elixirs, syrups, and the preferred forms of tablets, including chewable and orally disintegrating forms or suspensions. The compositions of the present invention may also be prepared for parenteral administration in the form of sterile suspensions for the injection or lyophilized powders for injection following reconstitution to a suspension. Administration by any other known route is also contemplated, such as transmucosally, transdermally, intravenously, intramuscularly, or intraparenterally.

Tablets containing the unique active combination of the present invention are prepared in a conventional manner by the addition of suitable pharmaceutical carriers including fillers, diluents, lubricants and the like as well as conventional and well known binding and disintegrating agents. Each tablet comprises, or consists essentially of, about 2 to 12 mg of chlorpheramine tannate, about 2 to 50 mg of phenylephrine tannate, and/or about 0.5 to 10 mg of methscopolamine tannate alone. The compositions may also comprise a therapeutic amount of another pharmaceutically active ingredient.

Suspensions comprising the unique active combination of the present invention are prepared by conventional well known compounding techniques and included various excipients as appropriate to the formulation.

### EXAMPLE 1

Preparation of Chlorpheniramine Tannate, Phenylephrine Tannate, and Methscopolamine Tannate Tablets

| Ingredient | Milligrams per Tablet | |
|---|---|---|
| Chlorpheniramine Tannate | 3.5 | mg |
| Phenylephrine Tannate | 25.0 | mg |
| Methscopolamine Tannate | 3.0 | mg |
| Compressible Sugar, NF | 399.5 | mg |
| Sodium Saccharin, USP | 1.0 | mg |
| Sodium Starch Glycolate, NF | 50.0 | mg |
| Magnasweet 100 | 5.0 | mg |
| FD&C Blue #2 aluminum lake | 1.5 | mg |
| FD&C Red #40 aluminum lake | 1.5 | mg |
| Artificial grape flavor | 5.0 | mg |
| Talc, USP | 5.0 | mg |

Tablets containing combinations of chlorpheramine tannate, phenylephrine tannate, methscopolamine tannate, and one or more additional active ingredients would comprise essentially the same ingredients in the same amounts. Changes in the additional active ingredient(s) present would be offset by the appropriate addition or subtraction to the compressible sugar amount. Total tablet weight would remain the same.

### EXAMPLE 2

Preparation of Chlorpheniramine Tannate, Phenylephrine Tannate, and Methscopolamine Tannate Suspension

Suspensions of the compositions of the present invention are prepared in a conventional manner such that each 5 mL (one teaspoon) would contain approximately 2 to 12 mg of chlorpheramine tannate, approximately 2 to 50 mg of phenylephrine tannate, and/or about 0.5 to 10 mg of methscopolamine tannate, alone or in combination with a therapeutic amount of another pharmaceutical active ingredient. Additionally, the suspension formulations may contain additional ingredients such as, but not limited to, citric acid, colorants, natural and artificial flavors, glycerin, magnesium aluminum silicate, methylparaben, propylparaben, purified water, sodium citrate, sweeteners such as sucralose, sucrose, or sorbitol, and xanthan gum. This example is illustrative of a typical suspension formulation of the present invention prepared by conventional well-known compounding techniques.

| Ingredient | Milligrams per 5 mL | |
|---|---|---|
| Chlorpheniramine Tannate | 3.5 | mg |
| Phenylephrine Tannate | 25.0 | mg |
| Methscopolamine Tannate | 3.0 | mg |
| Xanthan Gum, NF | 30.0 | mg |
| Magnesium Aluminum Silicate, NF | 35.0 | mg |
| Methylparaben, NF | 7.5 | mg |
| Propylparaben, NF | 1.5 | mg |
| Sucralose, NF | 7.5 | mg |
| Glycerin, USP | 250.0 | mg |
| Citric Acid, USP | 10.0 | mg* |
| Sodium Citrate, USP | 5.0 | mg* |
| Artificial Grape Flavor | 15.0 | mg |
| FD&C Blue #2 Dye | 7.2 | mg |
| FD&C Red #40 Dye | 7.2 | mg |
| Purified Water, USP (Deionized) | adjust to 5 | mL |

*Additional Citric Acid, USP or Sodium Citrate, USP, also may be included in the formula if needed for pH adjustment.

Suspensions containing combinations of chlorpheramine tannate, phenylephrine tannate, methscopolamine tannate, and one or more additional active ingredients would comprise essentially the same ingredients in the same amounts. Changes in the additional active ingredient(s) present would be offset by the appropriate addition or subtraction to the purified water content.

For the purpose of this disclosure, the term “warm-blooded animal” is used to refer to a member of the animal kingdom possessed of a homeostatic mechanism and includes mammals and birds.

The dosage and administration will be dependent on the age, health, and weight of the recipient, kinds of concurrent treatment, if any, frequency of treatment, and effect desired.

It should be understood that the above examples are illustrative of the best mode only of the invention herein disclosed. Given the present disclosure, it is anticipated that numerous variations will occur to those skilled in the art. A latitude of modification, substitution, and change is intended and in some instances, some features of the invention will be employed without a corresponding use of other features.

What is claimed is:

1. A therapeutic composition for the treatment of symptoms in a warm blooded animal in need of such treatment, wherein the symptoms are associated with a condition selected from the group consisting of the common cold, sinusitis, allergic rhinitis, respiratory congestion, other upper respiratory tract conditions, allergic skin reactions, urticaria, and angioedema; and wherein said composition comprises pharmaceutically effective amounts of methscopolamine tannate, alone or in combination with an antihistamine, decongestant, or combination thereof.

2. The therapeutic composition of claim 1, wherein said composition is a chewable tablet form or an orally disintegrating tablet form.

3. The therapeutic composition of claim 1, wherein said composition is in suspension form.

4. The therapeutic composition of claim 1, wherein said composition is in a liquid filled hard gelatin capsule form or a soft gelatin capsule form.

5. The therapeutic composition of claim 1, wherein the antihistamine is selected from the group consisting of chlorpheniramine tannate, dexchlorpheniramine tannate, brompheniramine tannate, dexbrompheniramine tannate, carbinoxamine tannate, pyrilamine tannte, and diphenhydramine tannate.

6. The therapeutic composition of claim 1, wherein the decongestant is selected from the group consisting of phenylephrine tannate and pseudoephedrine tannate.

7. A therapeutic composition for the treatment of symptoms in a warm blooded animal in need of such treatment, wherein the symptoms are associated with a condition selected from the group consisting of the common cold, sinusitis, allergic rhinitis, respiratory congestion, other upper respiratory tract conditions, allergic skin reactions, urticaria, and angioedema; wherein said composition comprises pharmaceutically effective amounts of methscopolamine tannate, alone or in combination with an antihistamine, decongestant, or combination thereof; and wherein said composition comprises pharmaceutically effective amounts of about 2 to 12 mg chlorpheniramine tannate, about 2 to 50 mg phenylephrine tannate, and about 0.5 to 10 mg methscopolamine tannate.

8. The therapeutic composition of claim 7, wherein said composition comprises pharmaceutically effective amounts of about 3.5 mg chlorpheniramine tannate, about 25 mg phenylephrine tannate, and about 3 mg methscopolamine tannate.

9. The therapeutic composition of claim 7, wherein said composition is a chewable tablet form or an orally disintegrating tablet form.

10. The therapeutic composition of claim 7, wherein said composition is in suspension form.

11. The therapeutic composition of claim 7, wherein said composition is in a liquid filled hard gelatin capsule form or a soft gelatin capsule form.

12. A method for treating and relieving symptoms associated with a condition in a warm-blooded animal over an extended period of time, wherein the condition is selected from a group consisting of the common cold, sinusitis, allergic rhinitis, respiratory congestion, other upper respiratory tract conditions, allergic skin reactions, urticaria, and angioedema, and wherein the method comprises orally administering to a warm-blooded animal in need of such treatment a pharmaceutically effective amount of a composition comprising pharmaceutically effective amounts of methscopolamine tannate, alone or in combination with an antihistamine, decongestant, or combination thereof.

13. The method of claim 12, wherein the antihistamine is selected from the group consisting of chlorpheniramine tannate, dexchlorpheniramine tannate, brompheniramine tannate, dexbrompheniramine tannate, carbinoxamine tannate, pyrilamine tannte, and diphenhydramine tannate.

14. The method of claim 12, wherein the decongestant is selected from the group consisting of phenylephrine tannate and pseudoephedrine tannate.

15. The method of claim 12, wherein said composition comprises pharmaceutically effective amounts of about 2 to 12 mg chlorpheniramine tannate, about 2 to 50 mg phenylephrine tannate, and about 0.5 to 10 mg methscopolamine tannate.

16. The method of claim 15, wherein said composition comprises pharmaceutically effective amounts of about 3.5 mg chlorpheniramine tannate, about 25 mg phenylephrine tannate, and about 3 mg methscopolamine tannate.

17. The method of claim 12, wherein said composition is in a chewable tablet form or an orally disintegrating tablet form.

18. The method of claim 12, wherein said composition is in a liquid filled hard gelatin capsule form or a soft gelatin capsule form.

19. The method of claim 12, wherein said composition is in a suspension form.

20. The method of claim 15, wherein said composition is in a chewable tablet form or an orally disintegrating tablet form.

21. The method of claim 15, wherein said composition is in a liquid filled hard gelatin capsule form or a soft gelatin capsule form.

22. The method of claim 15, wherein said composition is in a suspension form.
